(19) United States
(12) Patent Application Publication
(10) Pub. No.: US 2005/0209593 A1
(43) Pub. Date: Sep. 22, 2005

(54) Flexible anterior cervical plate

(75) Inventor: Eric Kolb, Quincy, MA (US)

Correspondence Address: PHILIP S. JOHNSON;JOHNSON & JOHNSON, ONE JOHNSON & JOHNSON PLAZA, NEW BRUNSWICK, NJ, 08933-7003, US

(73) Assignee: DePuy Spine, Inc., Raynham, MA (US)

(21) Appl. No.: 10/793,966

(22) Filed: Mar. 6, 2004

(51) Int. Cl. A61B 17/56

(52) U.S. Cl. 606/61

(57) ABSTRACT

An anterior cervical plate adapted for lateral flexion.

### BACKGROUND OF THE INVENTION

For a number of known reasons, bone fixation devices are useful for promoting proper healing of injured or damaged vertebral bone segments caused by trauma, tumor growth, or degenerative disc disease. The external fixation devices immobilize the injured bone segments to ensure the proper growth of new osseous tissue between the damaged segments. These types of external bone fixation devices often include internal bracing and instrumentation to stabilize the spinal column to facilitate the efficient healing of the damaged area without deformity or instability, while minimizing any immobilization and post-operative care of the patient.

One such device is an osteosynthesis plate, more commonly referred to as a bone fixation plate, that can be used to immobilize adjacent skeletal parts such as bones. Typically, the fixation plate is a rigid metal or polymeric plate positioned to span bones or bone segments that require immobilization with respect to one another. The plate is fastened to the respective bones, usually with bone screws, so that the plate remains in contact with the bones and fixes them in a desired position. Bone plates can be useful in providing the mechanical support necessary to keep vertebral bodies in proper position and bridge a weakened or diseased area such as when a disc, vertebral body or fragment has been removed.

Such plates have been used to immobilize a variety of bones, including vertebral bodies of the spine. These bone plate systems usually include a rigid bone plate having a plurality of screw openings. The openings are either holes or slots to allow for freedom of screw movement. The bone plate is placed against the damaged vertebral bodies and bone screws are used to secure the bone plate to the spine, usually with the bone screws being driven into the vertebral bodies. Exemplary systems like the one just described can be found in U.S. Pat. No. 6,159,213 to Rogozinski, U.S. Pat. No. 6,017,345 to Richelsoph, U.S. Pat. No. 5,676,666 to Oxland et al., U.S. Pat. No. 5,616,144 to Yapp et al., U.S. Pat. No. 5,549,612 to Yapp et al., U.S. Pat. No. 5,261,910 to Warden et al., and U.S. Pat. No. 4,696,290 to Steffee.

When it is desirable to stabilize the cervical portion of the spine, a fixation plate is often fixed to the anterior portion of the cervical vertebrae. Anteriorly-disposed cervical plates are typically classified by the method by which the device limits the motion of the bone screws in one vertebral body relative to the next. In general, the device fits into one of three classifications: rigid (no motion allowed); semi-rigid (toggling of the screw is allowed), and dynamic (unrestricted motion along the axis of the spine). The surgeon typically selects a device from one of these three classes based upon the specific needs of the patient.

One cause of cervical pain arises from rupture or degeneration of lumbar intervertebral discs. Neck pain may be caused by the compression of spinal nerve roots by damaged discs between the vertebrae. One conventional method of managing this problem is to remove the problematic disc and fuse the adjacent vertebrae. Typically, the fusion is facilitated by filling the intevertebral disk space with autograft bone graft (such as bone chips) which contain matrix molecules and living cells such as osteoblasts which facilitate fusion. However, failure to distribute loads through the graft has been associated with an elevated incidence of non-unions.

Dynamic cervical plates address the loading problem and provide the benefit of allowing a continuous loading of the interbody graft even if some measure of graft subsidence has occurred. U.S. Pat. No. 6,669,700 (“Farris”) discloses a rigid anterior cervical plate having overlapping central screw holes. However, the rigidity of such plates may not allow for desirable continuous loading of the graft.

Although conventional dynamic plates desirably provide continuous loading, there are a number of issues related to such conventional dynamic plates. Some of these systems are characterized by multiple components, wherein dynamism is provided by the sliding of a superior component upon two axially disposed rods. Other systems are characterized by plates having slots that allow the associated bone fixation screws to translate and toggle related to the rod. However, some of these dynamic plates may intrude upon the adjacent disc space. In addition, the multiplicity of components in some of these dynamic plate systems requires a complex assembly.

U.S. Pat. No. 6,206,882 (“Cohen”) discloses a cervical plate having laterally extending slots, thereby allowing the surgeon to easily bend the plate at the time of surgery so that it may conform to the patient's anatomy. Since the slots are either strictly lateral or diagonal and open onto the lateral edges of the plate, the Cohen plate can easily be twisted or bent. However, the Cohen plate does not provide axial displacement.

U.S. Published Patent Application No. 2003/0229348 (“Sevrain”) discloses a connecting device for attaching at least two adjacent vertebrae. FIGS. 1, 1a-1c each disclose a device having joint 16 having an apex 22. This joint provides a springing action that responds to natural flexion and extension. However, Sevrain further discloses that this device is adapted for use as a disc prosthesis, not as a fusion device. The devices in Sevrain that are to be used as fusion devices (e.g., FIGS. 7-10) appear to have no flexible portions.

### SUMMARY OF THE INVENTION

The present inventor has developed anterior cervical plates that address a number of the concerns described above.

In some embodiments of the present invention, there is provided a dynamic anterior cervical plate that will not interfere with adjacent discs and requires only a single plate component (excluding screws and locking features) to achieve continuous graft loading.

In particular, the present invention relates to an anterior cervical plate having an intermediate elongated portion that flexes axially and laterally in response to an axial load. The axial flexion of this intermediate portion has the effect of reducing the distance between the upper and lower bone screws fixed to the adjacent vertebrae through the plate, thereby allowing the device to properly respond to a change in loading of the functional spinal unit. The lateral flexion of this intermediate portion insures that the device will not protrude anteriorly into critical organs or posteriorly into the graft.

Therefore, in accordance with the present invention, there is provided a cervical plate for providing dynamic stabilization of upper and lower cervical vertebrae, the plate having opposed inner and outer surfaces defining a transverse axis, and opposed upper and lower surfaces defining an elongated longitudinal axis, the plate comprising:
a) an upper portion having an upper transverse throughhole,
b) a lower portion having a lower transverse throughhole, and
c) a longitudinally elongated intermediate portion therebetween,
   wherein the elongated portion is adapted to flex laterally under loading of the longitudinal axis.

### DESCRIPTION OF THE FIGURES

FIG. 1 is a perspective view of a first embodiment of the present invention wherein axial flexibility is provided by a plurality of axially extending slots in the elongated intermediate portion.

FIG. 2 is a perspective view of a second embodiment of the present invention wherein the elongated intermediate portion has a pair of laterally flexible members.

FIG. 3 is a side view of the device of FIG. 2 implanted between two vertebrae.

FIG. 4 is a plan view of a third embodiment of the present invention having a telescoping stop and a plurality of bend zones.

FIG. 5a is a plan view of a fourth embodiment of the present invention having a contour zone.

FIG. 5b is a medial-lateral cross section of FIG. 5a.

FIGS. 6a and 6b are top views of an embodiment of the present invention adapted to flex laterally inward.

### DETAILED DESCRIPTION OF THE INVENTION

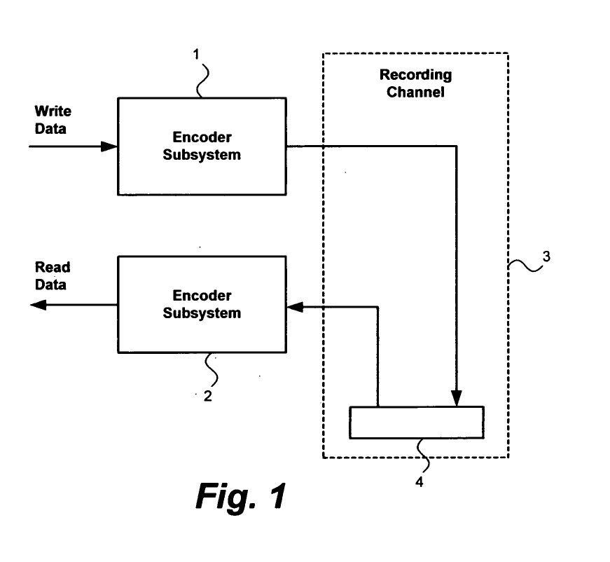

Now referring to FIG. 1, there is provided a cervical plate 1 for providing dynamic stabilization of upper and lower cervical vertebrae, the plate having opposed inner (not shown) and outer 5 surfaces defining a transverse axis, and opposed upper 7 and lower 9 surfaces defining an longitudinal axis L, the plate comprising:
a) an upper portion 11 having a pair of upper transverse throughholes 13,
b) a lower portion 15 having a pair of lower transverse throughholes 17, and
c) a longitudinally elongated intermediate portion 19 therebetween,
   wherein the elongated portion comprises:
i) a pair of lateral axially extending strut members 21, 23, each member extending from the upper portion to the lower portion and having a plurality of axially extending closed slots 25 therein, the slots defining a plurality of axially extending thin members within each axially extending strut member, and
ii) a large transverse hole 27 defining a graft window.

The plate component is composed of a generally flat piece of metal having at least one thin member oriented such that the thin member will deflect under application of a physiologic axial load. Deflection of the members decreases the hole-to-hole spacing of the plate, thus permitting continuous loading of the fusion graft. In this particular case, the intermediate portion of FIG. 1 flexes laterally outward.

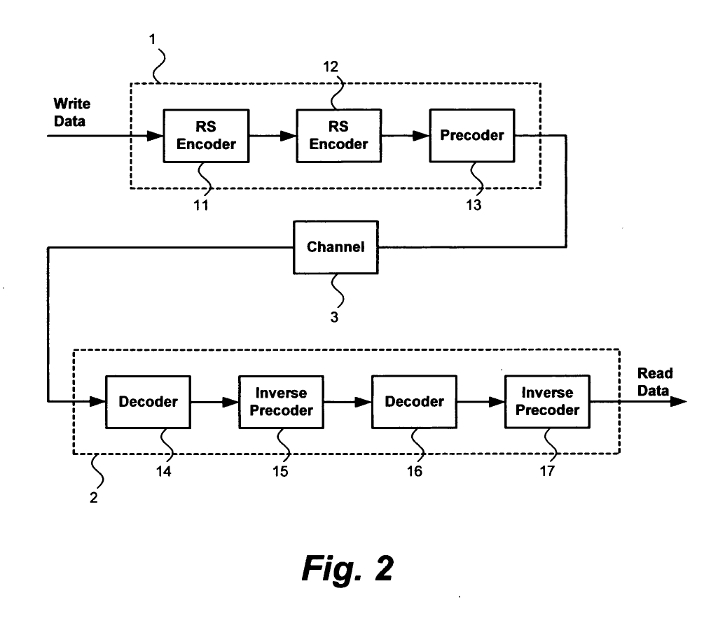

In some embodiments (as in FIG. 2), the elongated intermediate section comprises at least two flexible members extending from the upper to the lower portion of the device.

Now referring to FIG. 2, there is provided a cervical plate 31 for providing dynamic stabilization of upper and lower cervical vertebrae, the plate having opposed inner (not shown) and outer 35 surfaces defining a transverse axis, and opposed upper 37 and lower 39 surfaces defining an elongated longitudinal axis, the plate comprising:
a) an upper portion 41 having an upper transverse throughhole 43,
b) a lower portion 45 having a lower transverse throughhole 47, and
c) a longitudinally elongated intermediate portion 49 therebetween,
   wherein the elongated portion comprises:
i) a pair of lateral axially extending thin members 51 extending from the upper portion to the lower portion, each member having a straight portion 52 and a predetermined curve portion 53, and
ii) a telescoping portion 55.

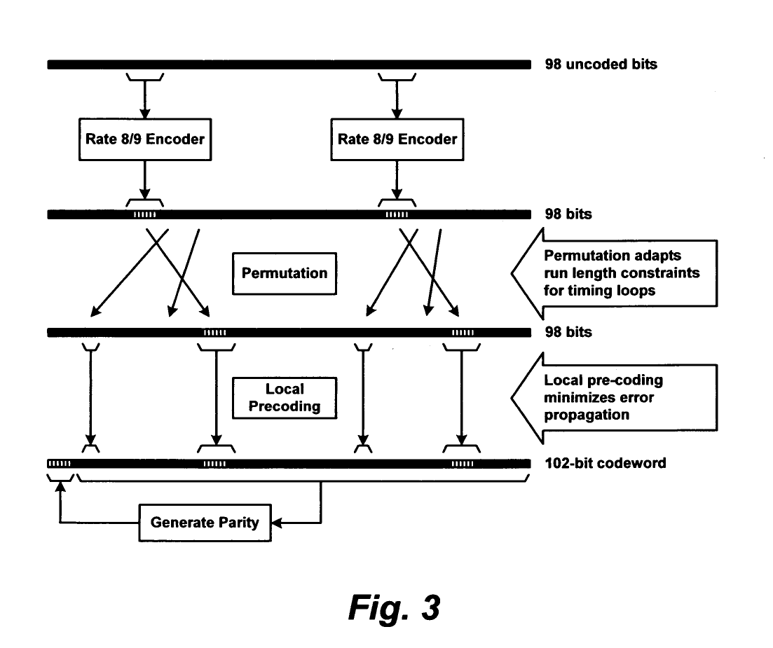

Now referring to FIG. 3, there is provided a side view of a device substantially similar to the device of FIG. 2 fixed between upper VB_{U} and lower VB_{L} vertebrae. The fixation creates a disc space into which a graft G is placed. The lateral flexing of the thin members prevents both anterior and posterior intrusion of the device.

In some embodiments, the elongated intermediate section comprises a number of axially extending slots that define a plurality of flexible members extending from the upper to the lower portion of the device. Preferably, the slotting of the device produces an even number of flexible members, thereby allowing uniform lateral flexing of the device. In some embodiments, as in FIG. 1, the slotting produces four thin members 28 per side. In some embodiments, the thin members have a combined width that comprises between about 10% and 30% of the width of the intermediate portion of the plate.

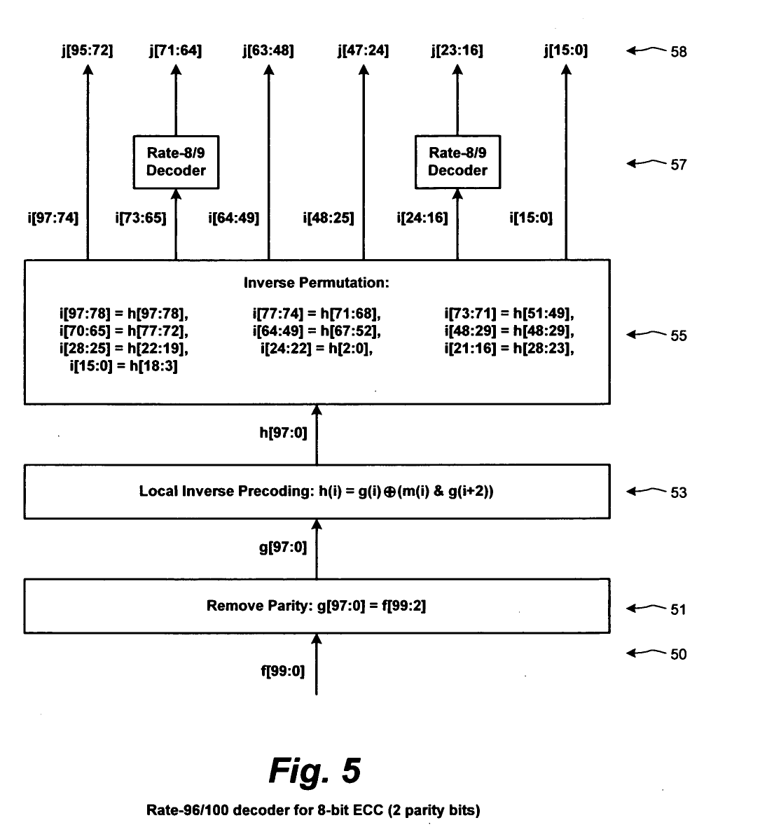

In some embodiments, the thin members have a generally uniform rectangular cross-section. In some preferred embodiments, and now referring to FIG. 5b, the long edge LE of the thin member extends in the anterior-posterior direction, while the short edge SE of the thin member extends in the medial-lateral direction. In this preferred embodiment, it is relatively easy to achieve motion in the axial direction, but difficult to deflect under flexion/extension and torsional loading.

Referring back to FIG. 2, in some embodiments, the thin flexible members extend (in an unloaded situation) axially along a curved portion 53. Since substantially straight thin members would not flex in the desired manner until a force sufficient to cause buckling were applied, resulting in an undesirable non-linear force-displacement curve. The pre-curved thin member of FIG. 2 will displace substantially linearly in response to an axial force, thereby providing the continuity of loading desirable for graft fusion.

Lateral flexing has an advantage over anterior flexing in that the lateral flex will not cause the thin member to touch any vital soft tissue organs such as the esophagus.

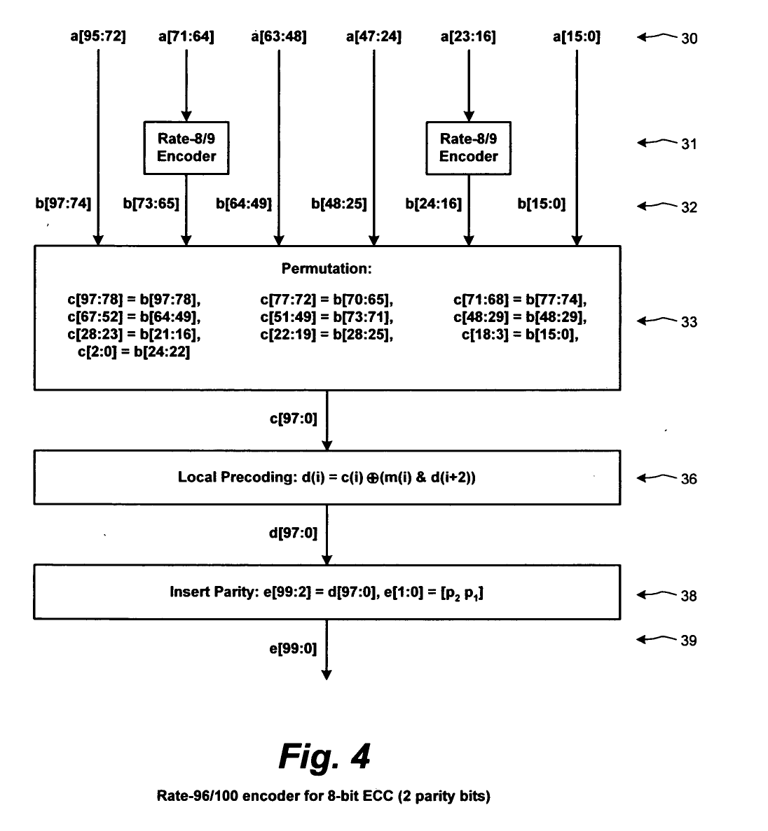

Now referring to FIG. 4, there is provided a cervical plate 81 for providing dynamic stabilization of upper and lower cervical vertebrae, the plate having opposed inner (not shown) and outer 85 surfaces defining a transverse axis, and opposed upper 87 and lower 89 surfaces defining an elongated longitudinal axis, the plate comprising:
a) an upper portion 91 having an upper transverse throughhole 93,
b) a lower portion 95 having a lower transverse throughhole 97, and
c) a longitudinally elongated intermediate portion 99 therebetween,
   wherein the elongated portion comprises:
i) a pair of lateral axially extending strut members 101, each strut member extending from the upper portion to the lower portion and having an upper thick-cross sectional portion 102, a lower thick-cross sectional portion 103, and a thin cross-sectional portion 104 therebetween, and
ii) a medially-located telescoping portion 115.

In some embodiments, axial flexibility is accomplished by providing a segment of reduced cross-section within the elongated intermediate portion. This segment produces a bend zone. Bend zones are desirable in that they provide some degree of plastic deformation and result in a kinked condition.).

Although the plates of the present invention provide an advantage in that they allow a measure of dynamism to provide continuous loading of the graft, a general goal of any such plate is still to provide a reasonable amount of stability to the graft site so as to prevent extreme subsidence and maintain the desired intervertebral spacing. Accordingly, in some embodiments of the present invention, the plate is further provided with a stop mechanism that prevents the hole-to-hole distance from falling below a predetermined value. Preferably, the stop limits the motion provided by the dynamic aspects of the plate to clinically significant values, such as the height of the disc space.

Still referring to FIG. 4, in these embodiments, the plate of the present invention includes a telescope unit 105 comprising:
a) a rod 107 extending from the upper portion and having a first end 108, and
b) a receiving tube 109 extending from the lower portion and having a bore 111 and a closed end surface 113.
   During acceptable levels of flexion, the rod simply translates within the receiving tube and does not affect the limits of axial motion. Under extreme axial load, however, the end 108 of the rod contacts the closed end surface 113 of the receiving tube, thereby preventing more axial displacement and preserving disc space height.

Although the telescoping unit of FIG. 4 is adapted to provide a stop in response to extreme motion, in other embodiments, the telescoping unit is adapted to simply provide relative sliding of the components and does not provide a stop.

The transverse holes (such as holes 115 of FIG. 4) through which the fasteners are fixed to the bone form a plurality of fastener-plate interfaces 116. Any conventional fastener-plate interface may be used in accordance with the present invention. In one preferred embodiment (as shown in FIG. 4), the interface 116 forms a snap-ring screw engagement. In some embodiments, the interface takes a form substantially similar to U.S. Pat. No. 4,493,317, the specification of which is incorporated by reference in its entirety.

In some embodiments, the device is provided with contour zones. The contour zones allow the surgeon to bend the device to accommodate for the lordotic curve of the cervical portion of the spine. In some embodiments, the contour zone is simply a thinned section disposed between an upper or lower portion and the intermediate portion.

Now referring to FIGS. 5a and 5b, there is provided a cervical plate 61 for providing dynamic stabilization of upper and lower cervical vertebrae, the plate having opposed inner (not shown) and outer 65 surfaces defining a transverse axis, and opposed upper 67 and lower 69 surfaces defining an elongated longitudinal axis, the plate comprising:
a. an upper portion 71 having a single upper transverse throughhole 73,
b. a lower portion 75 having a single lower transverse throughhole 77, and
c. a longitudinally elongated intermediate portion 79 therebetween, and
d. an upper contour zone 81 disposed between the upper portion and the intermediate portion, and
e. a lower contour zone 83 disposed between the lower portion and the intermediate portion.

In some embodiments, the elongated intermediate portion of the plate has a large transverse throughhole 85. This hole acts as a graft window, thereby allowing visualization of the graft throughout the plating procedure. In some embodiments, the width of the graft window is such that the strut members collectively comprise between about 10% and 40% of the total width W of the intermediate portion of the plate. In some embodiments, the width of the graft window comprises at least 30% of the toal width W of the longitudinally elongated intermediate portion.

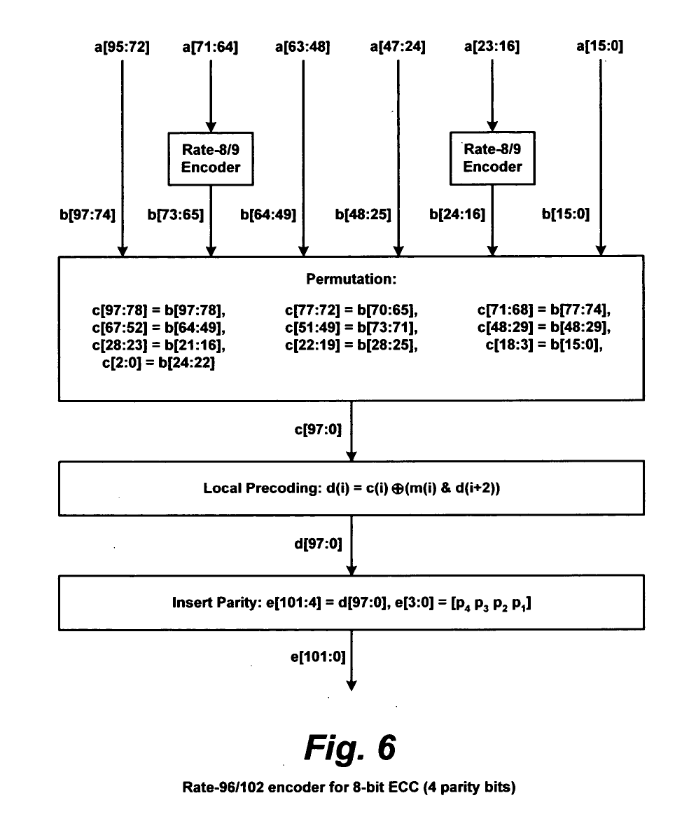

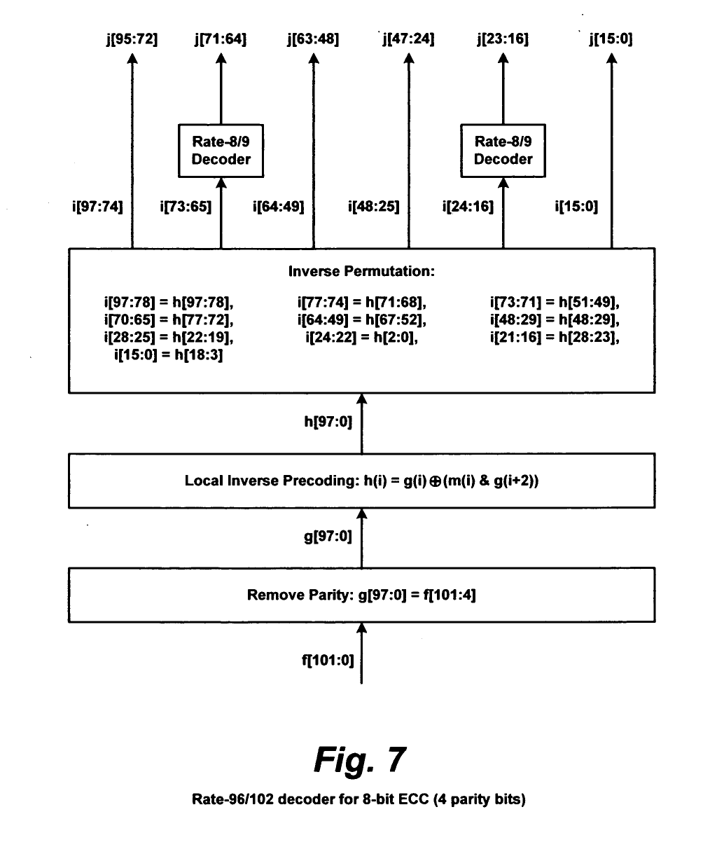

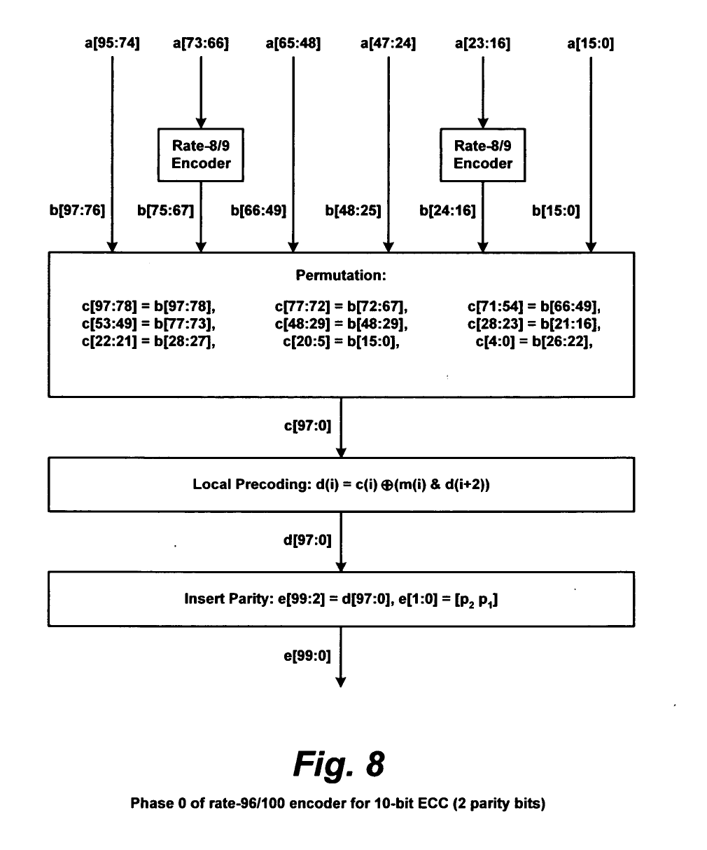

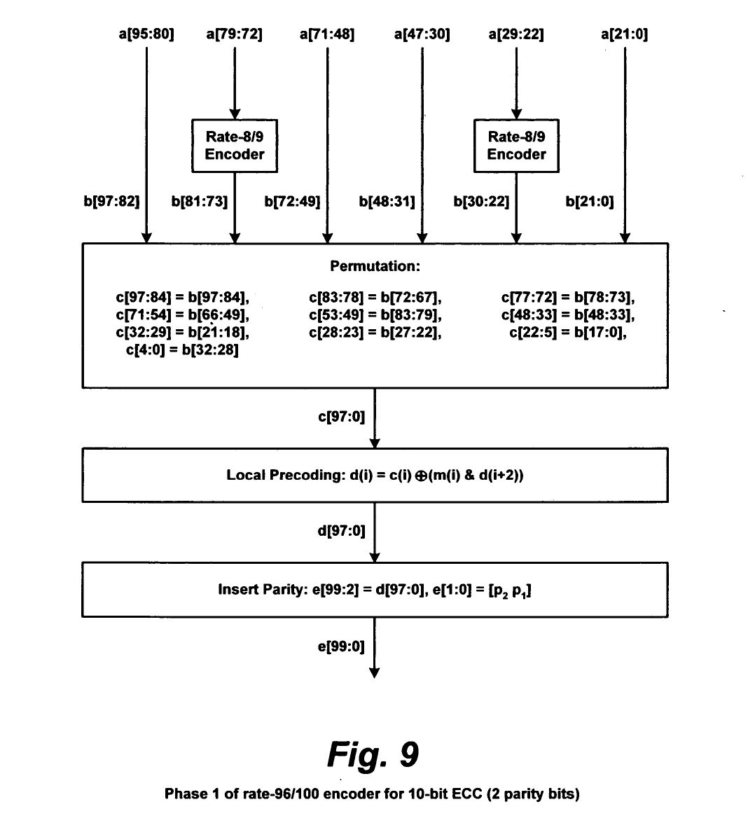

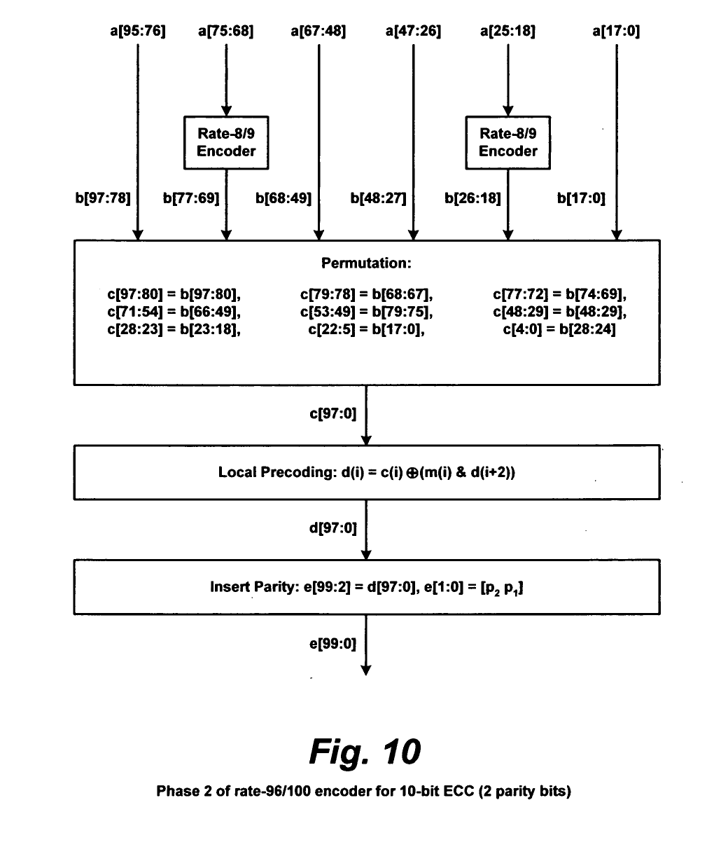

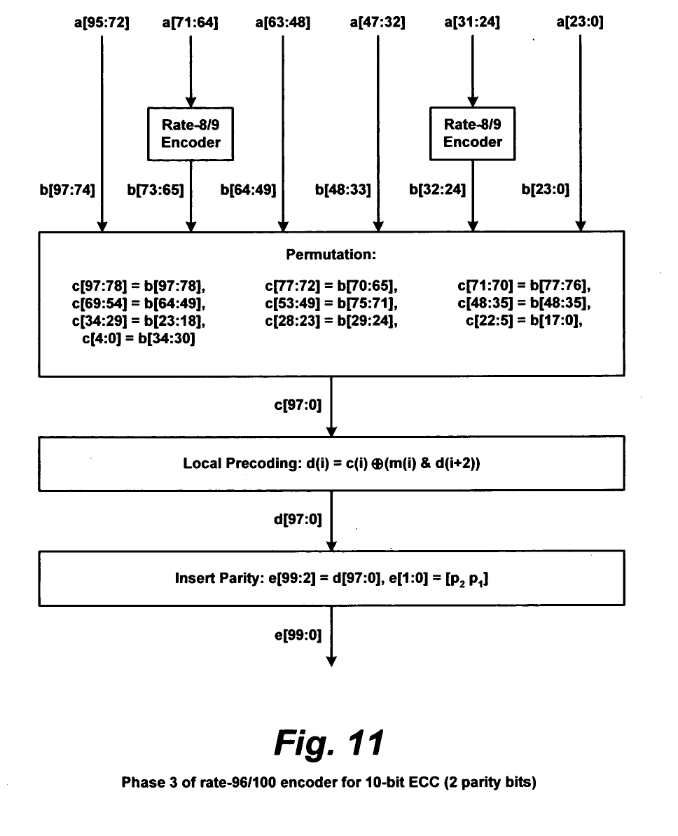

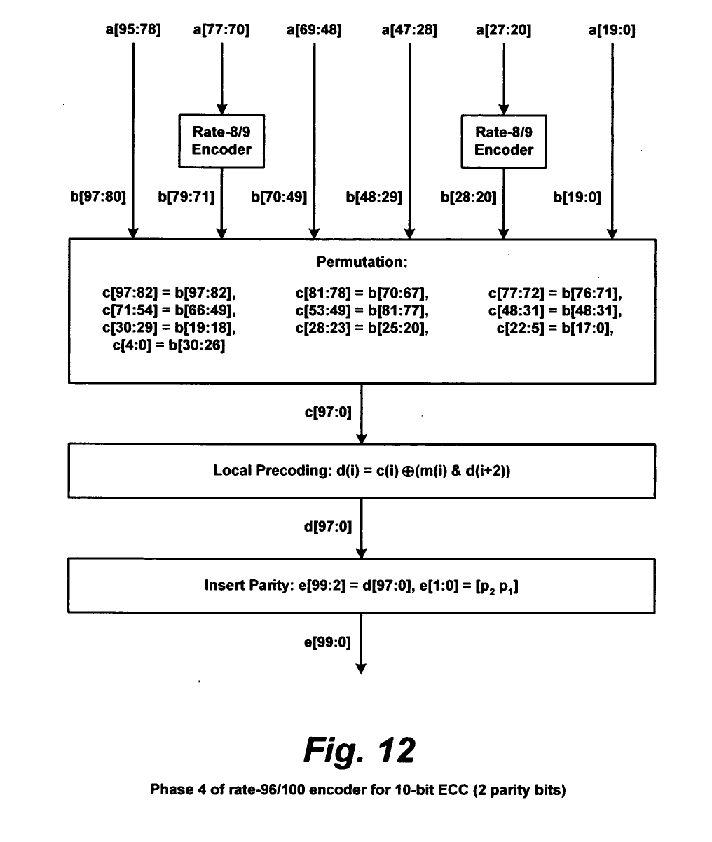

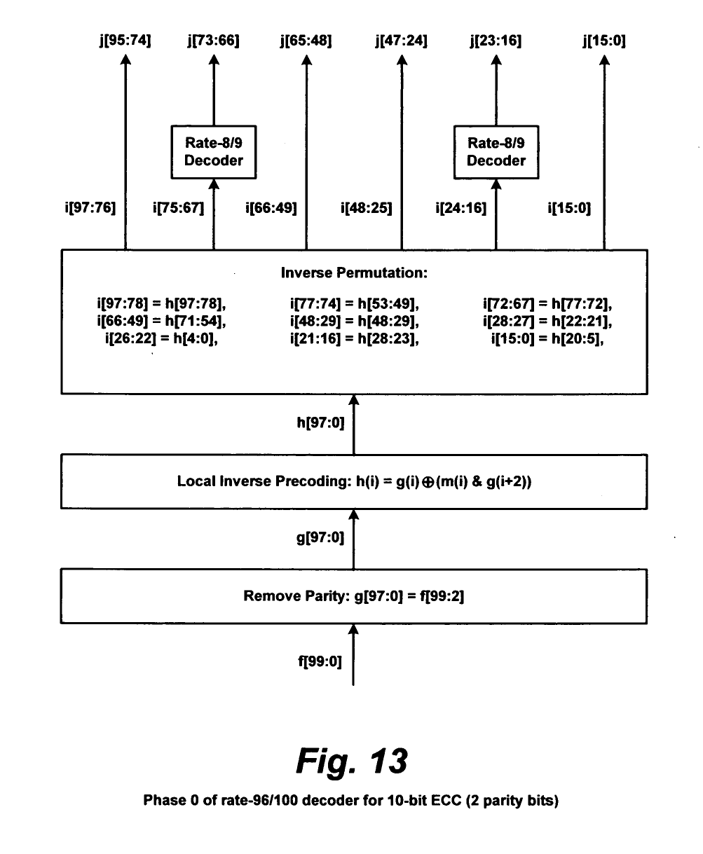

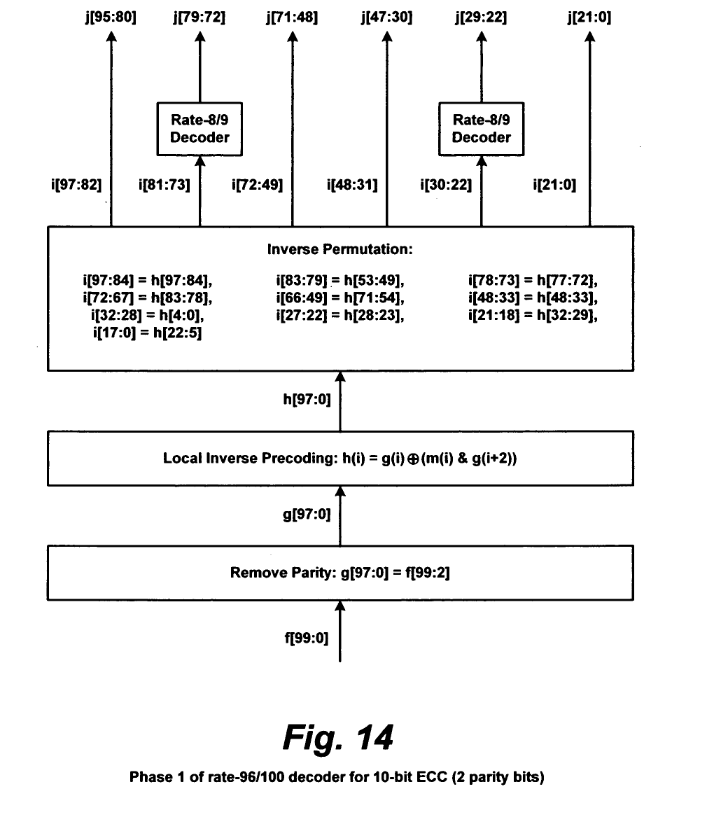

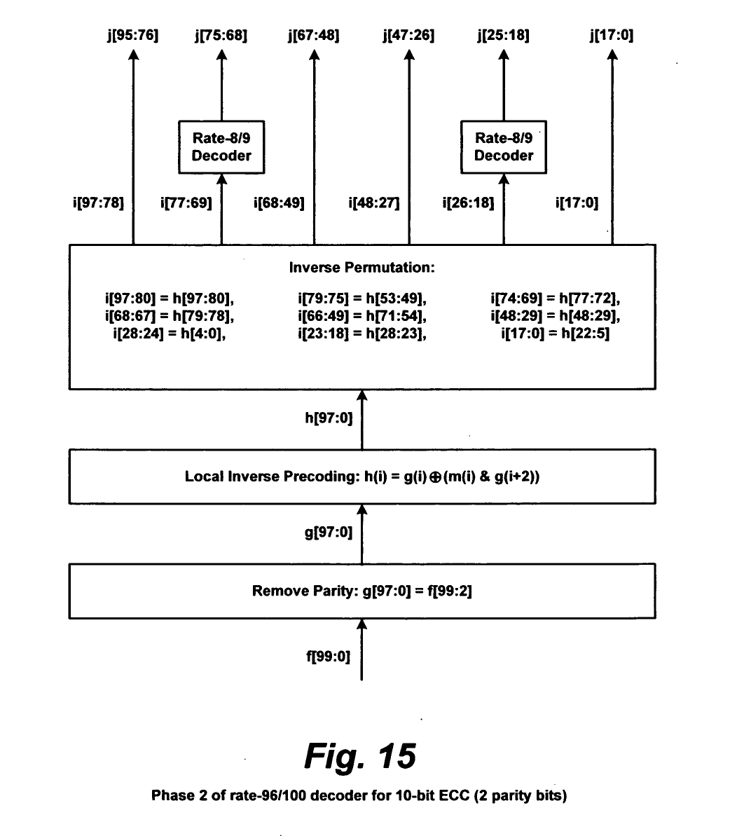

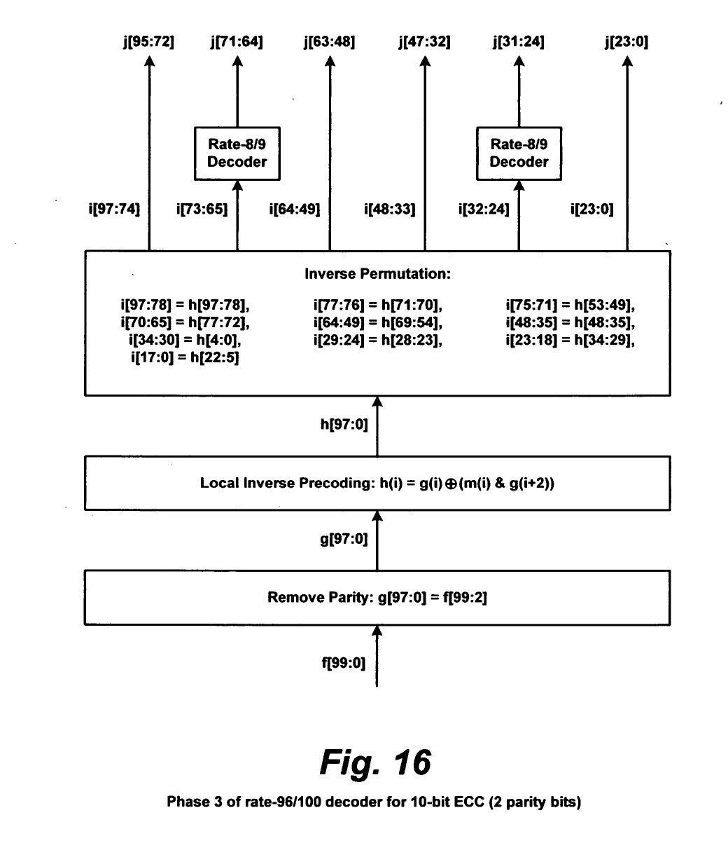

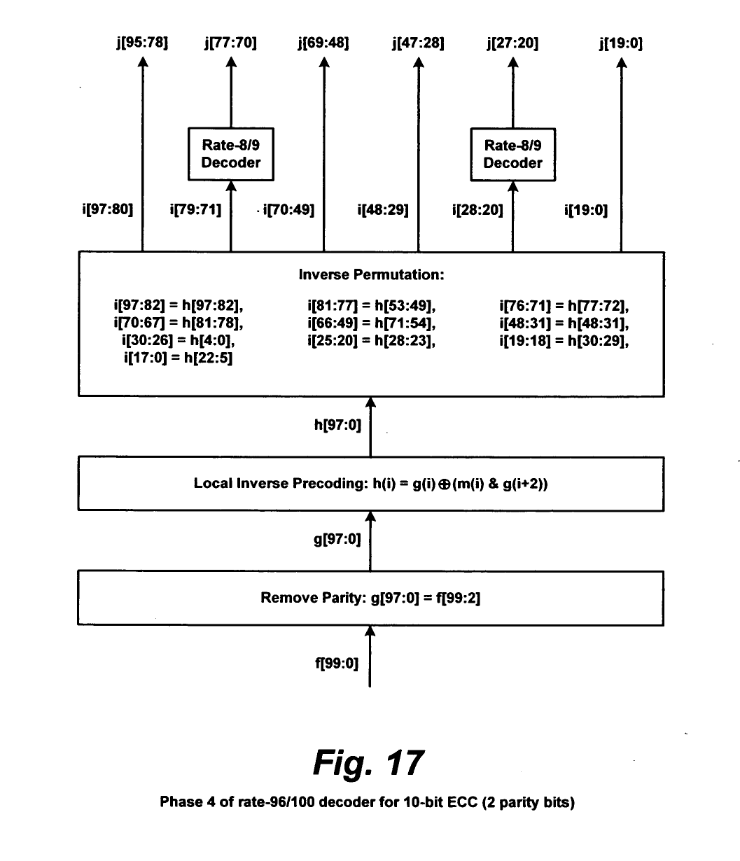

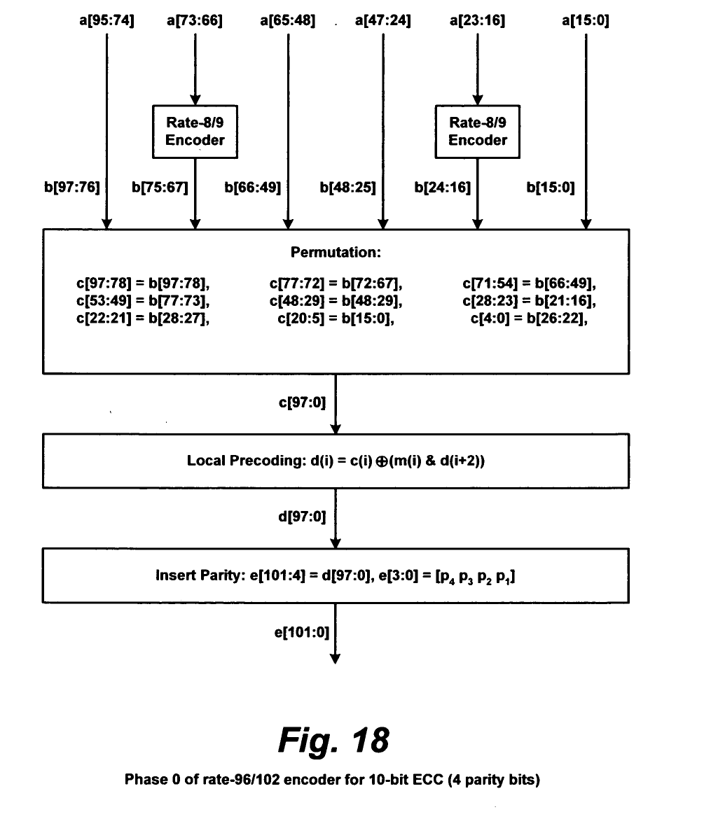

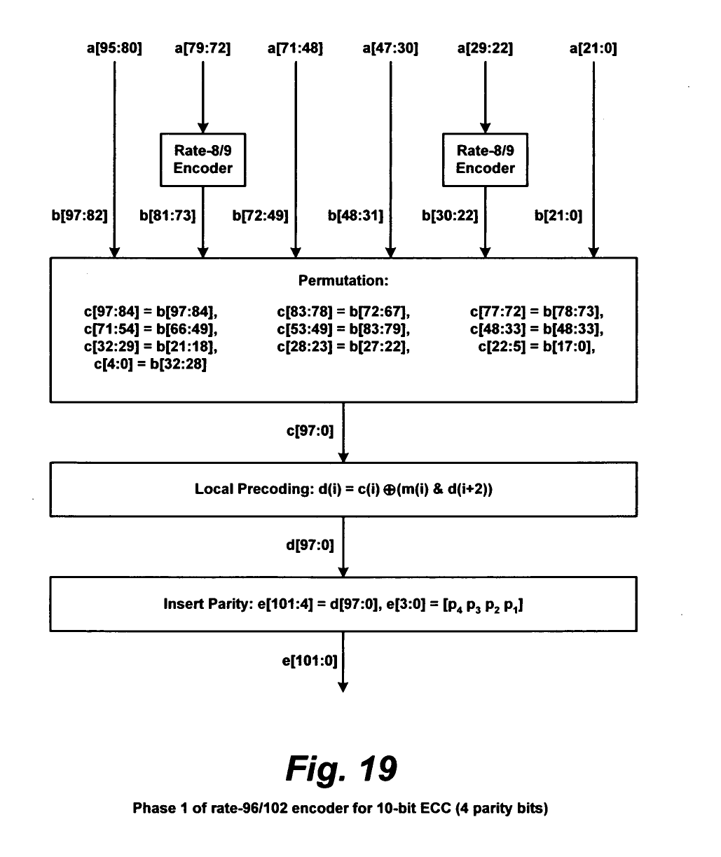

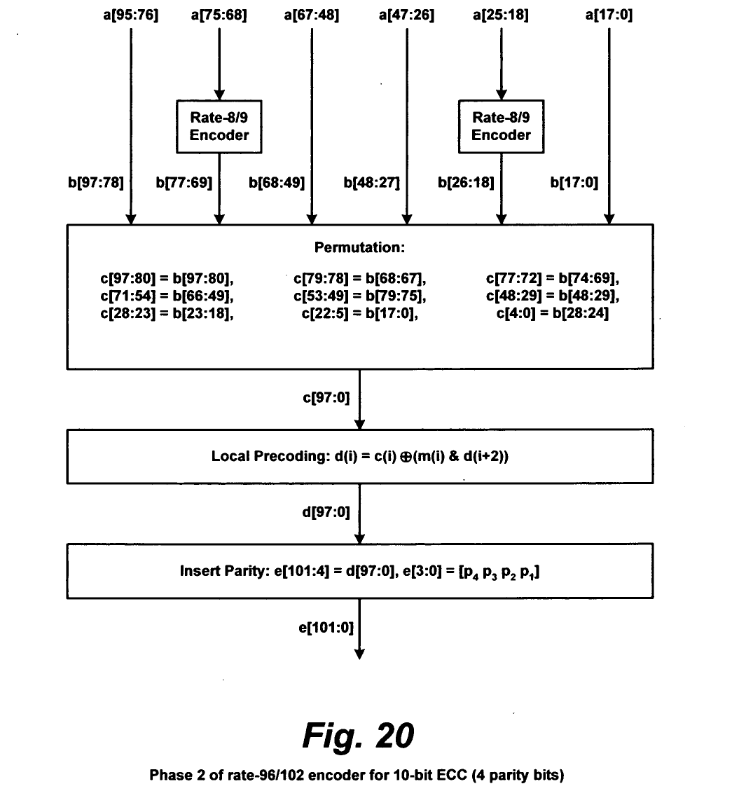

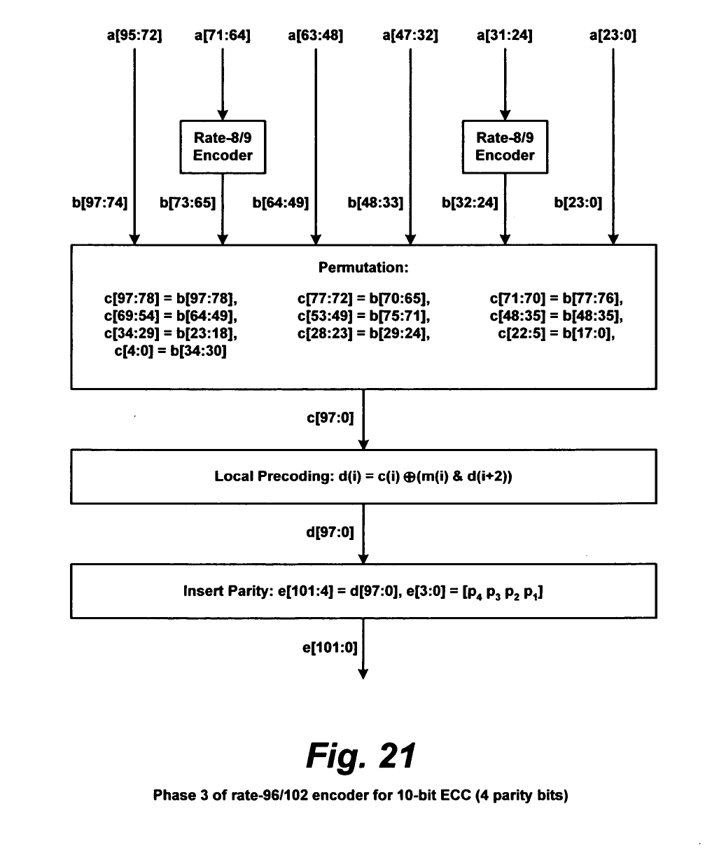

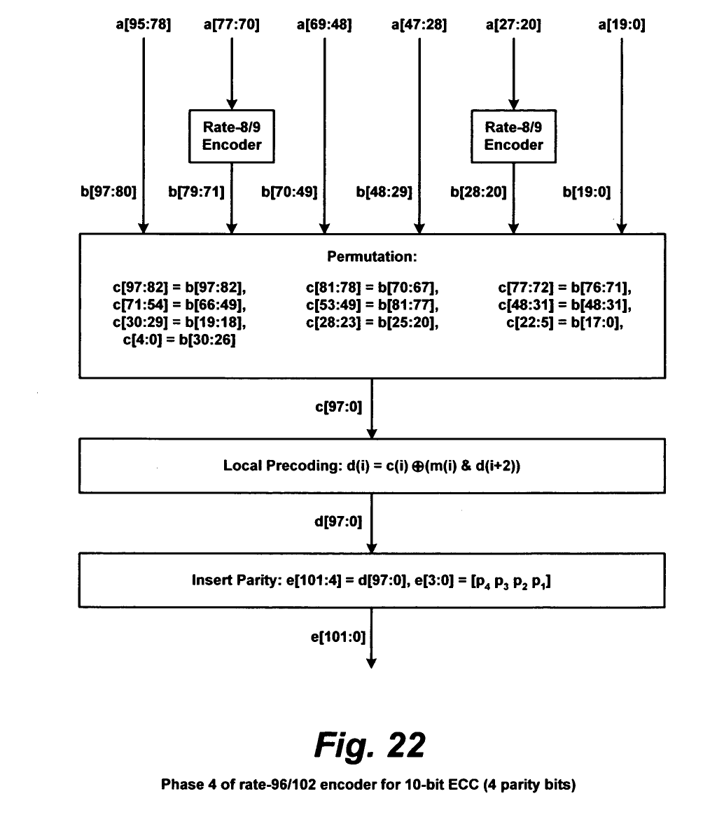

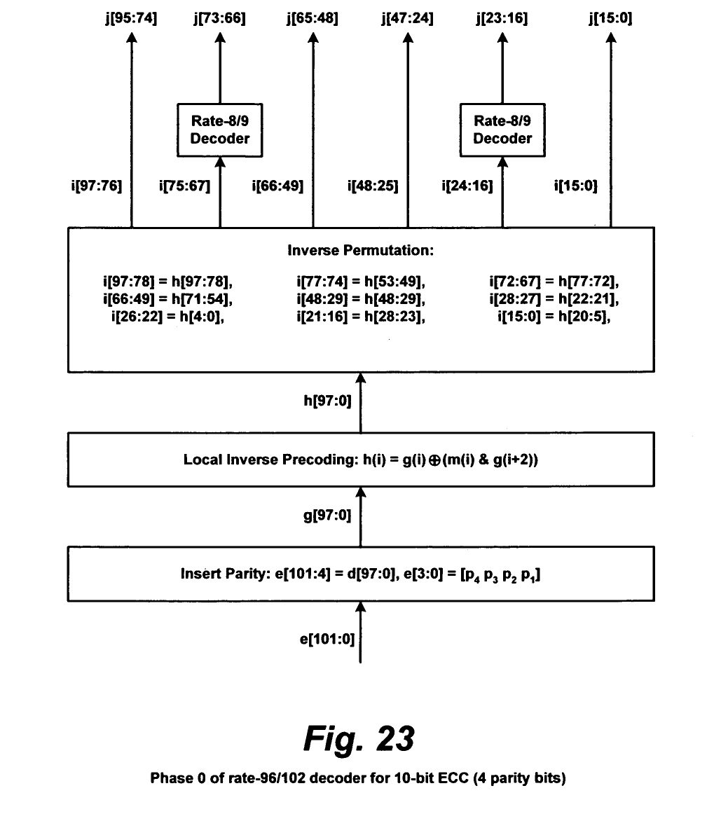

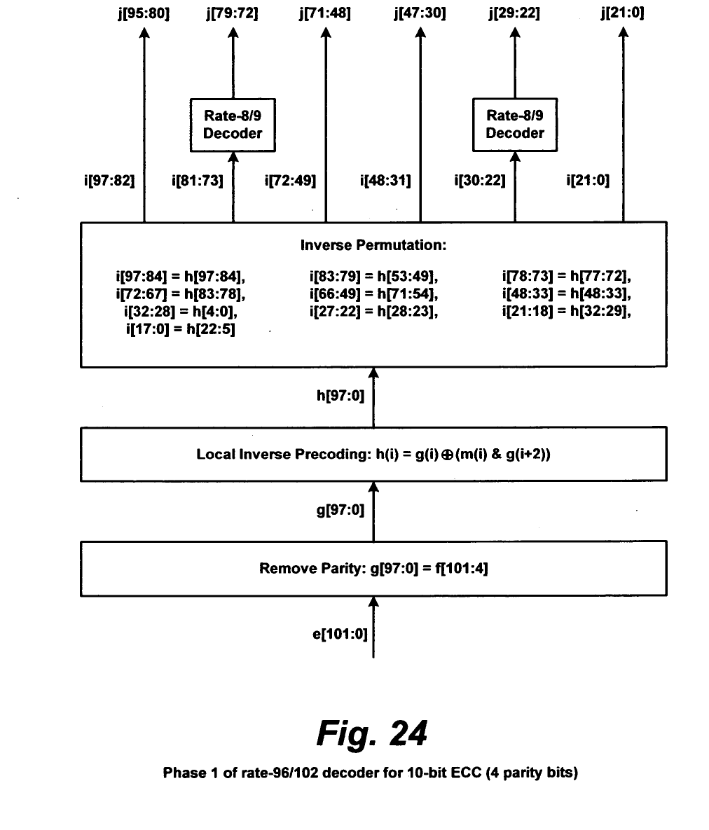

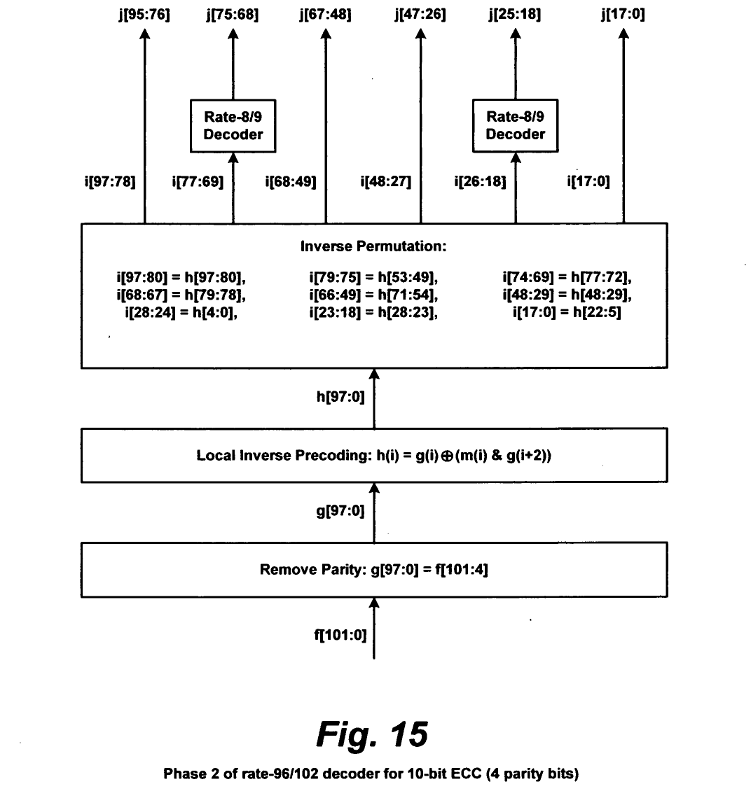

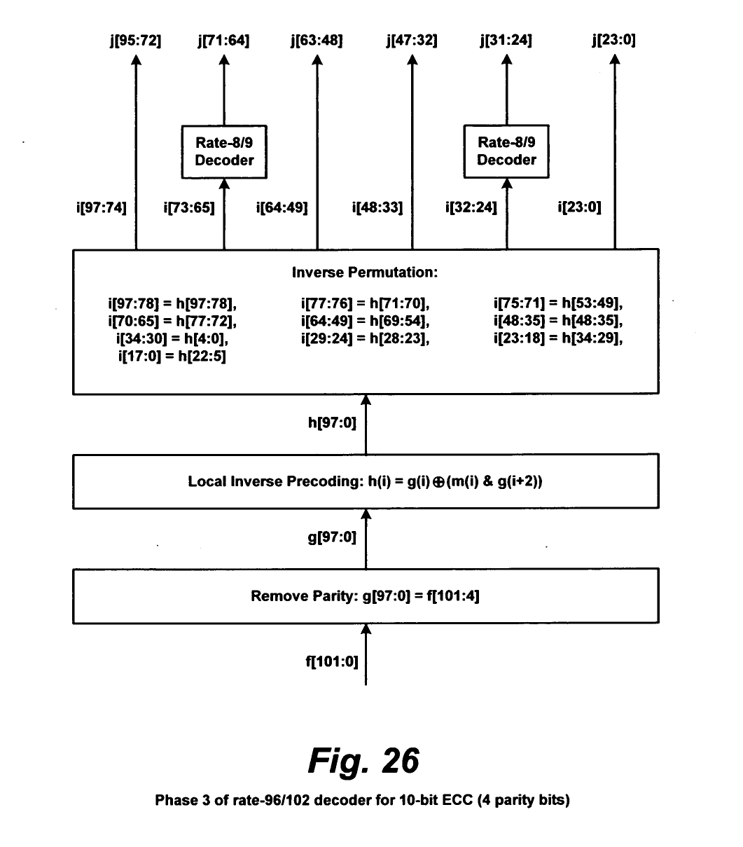

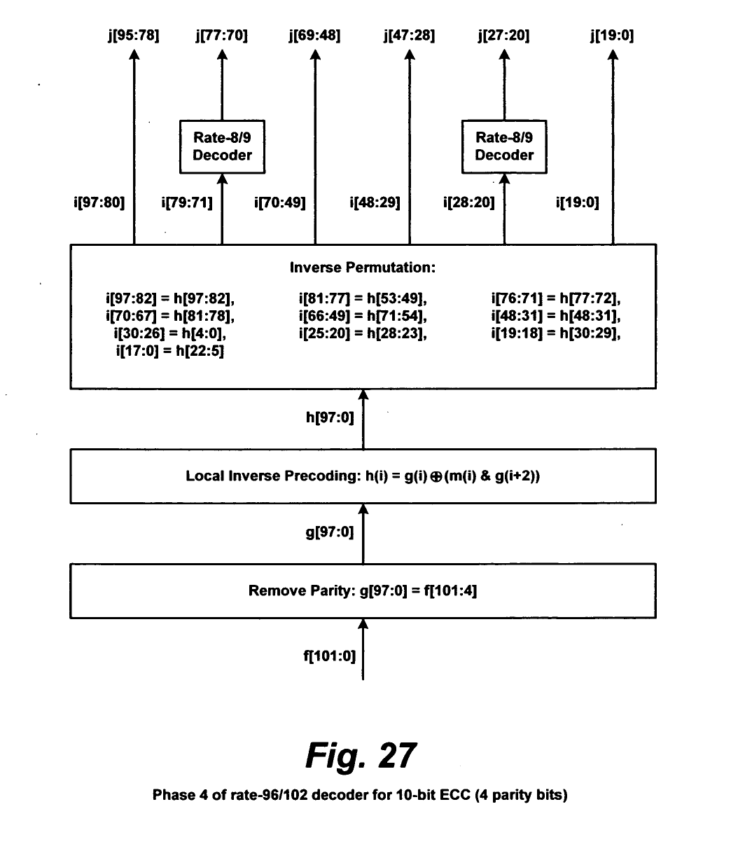

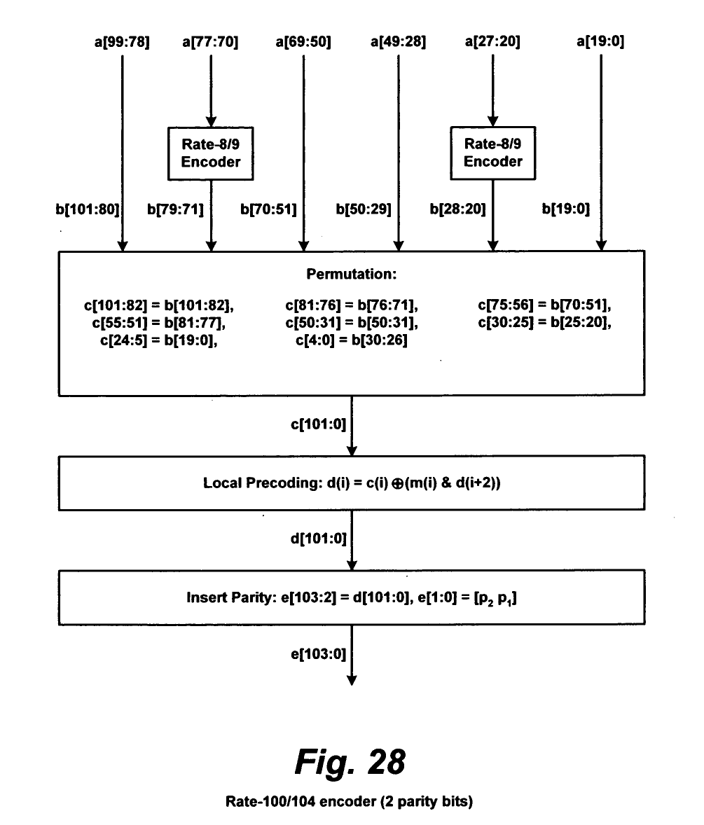

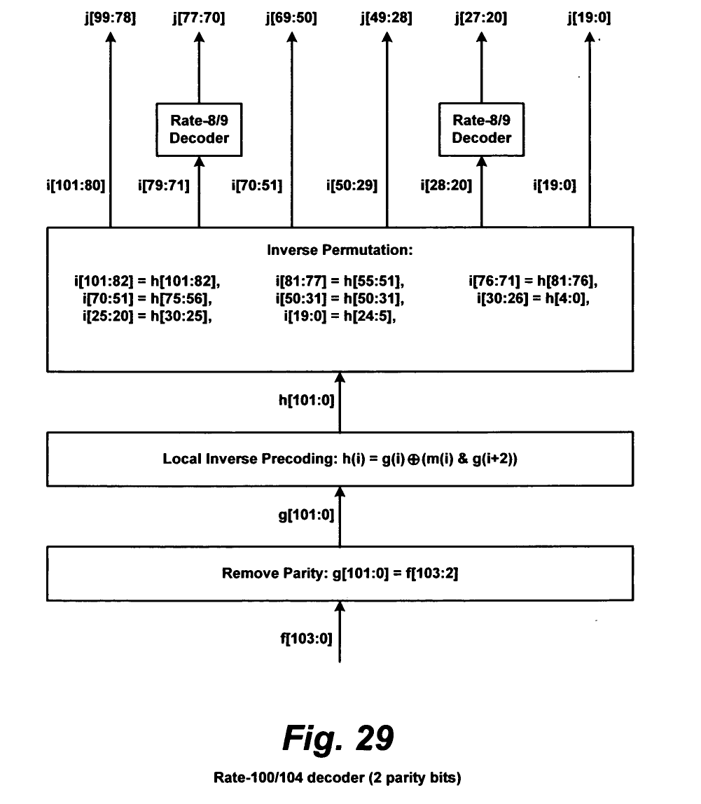

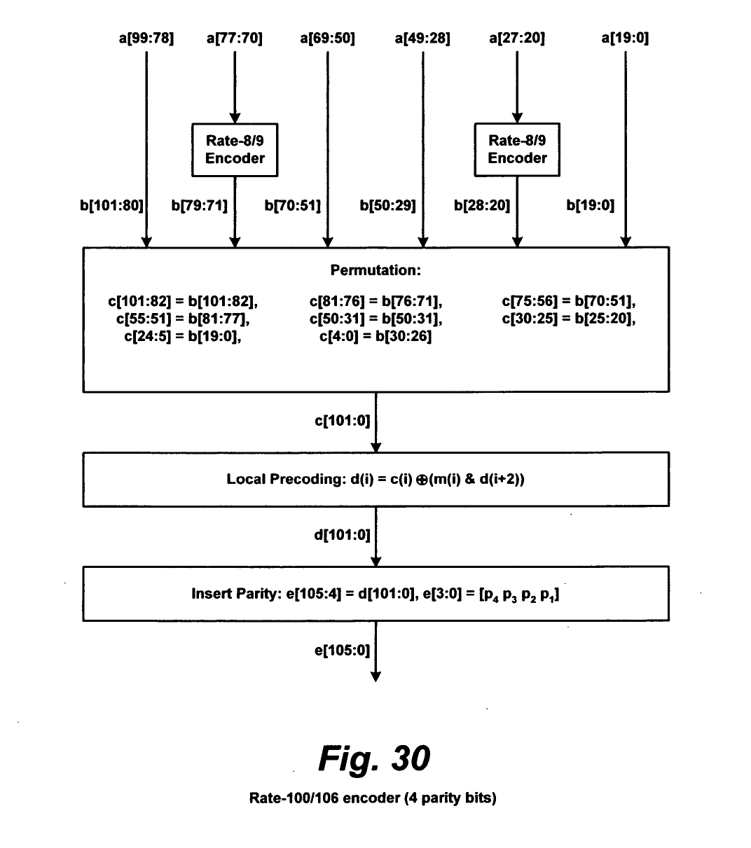

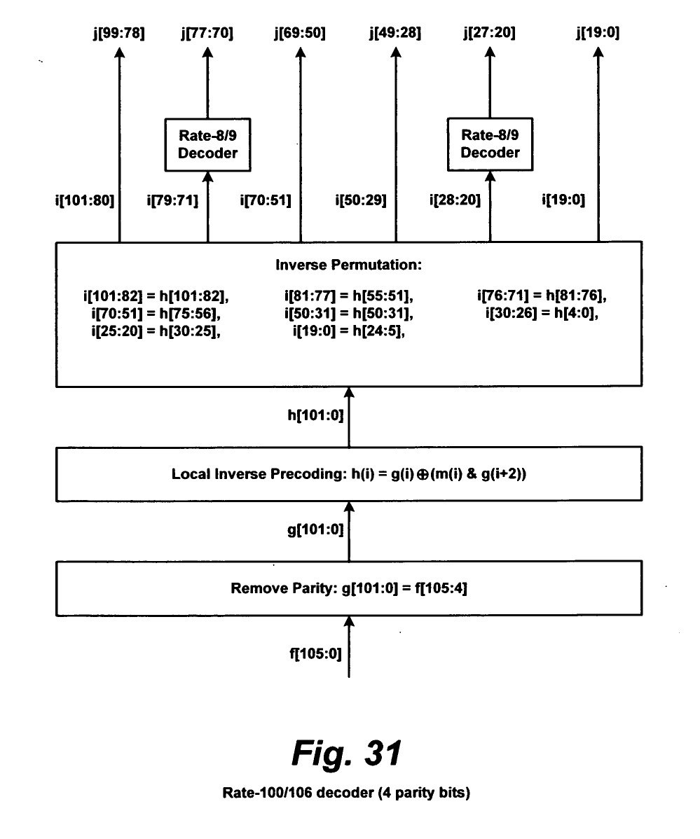

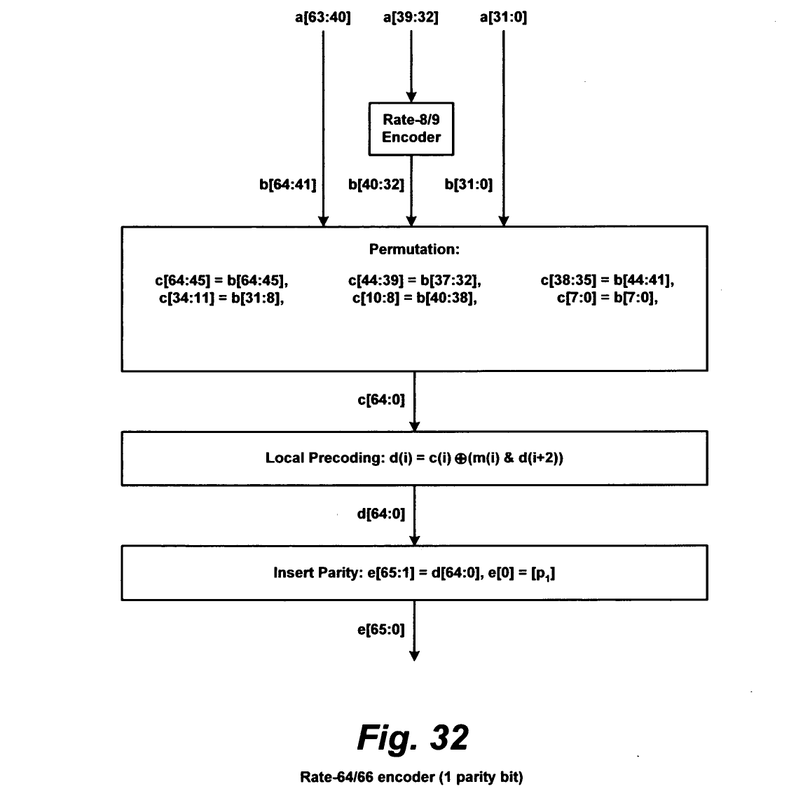

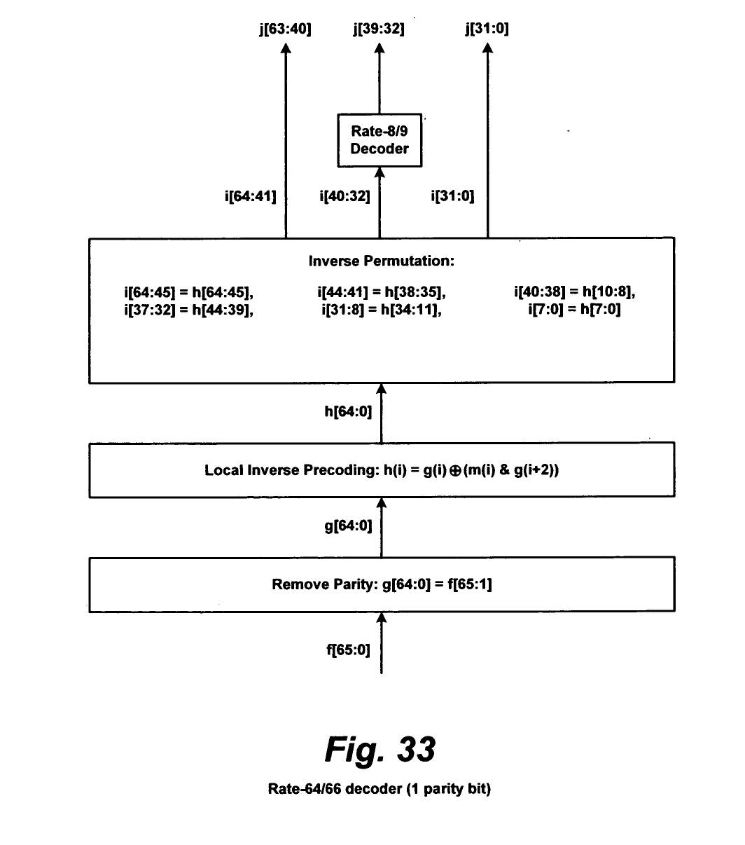

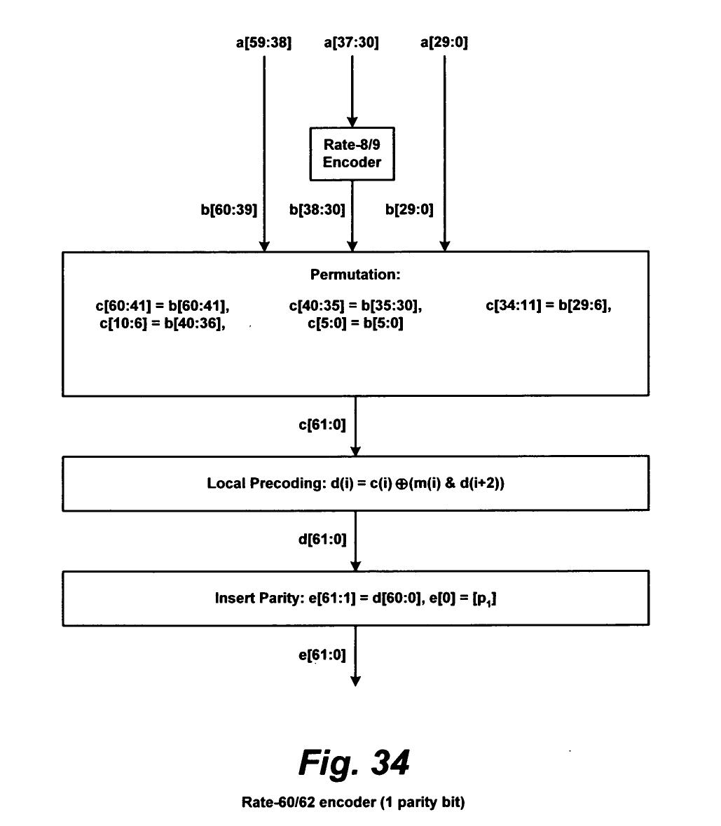

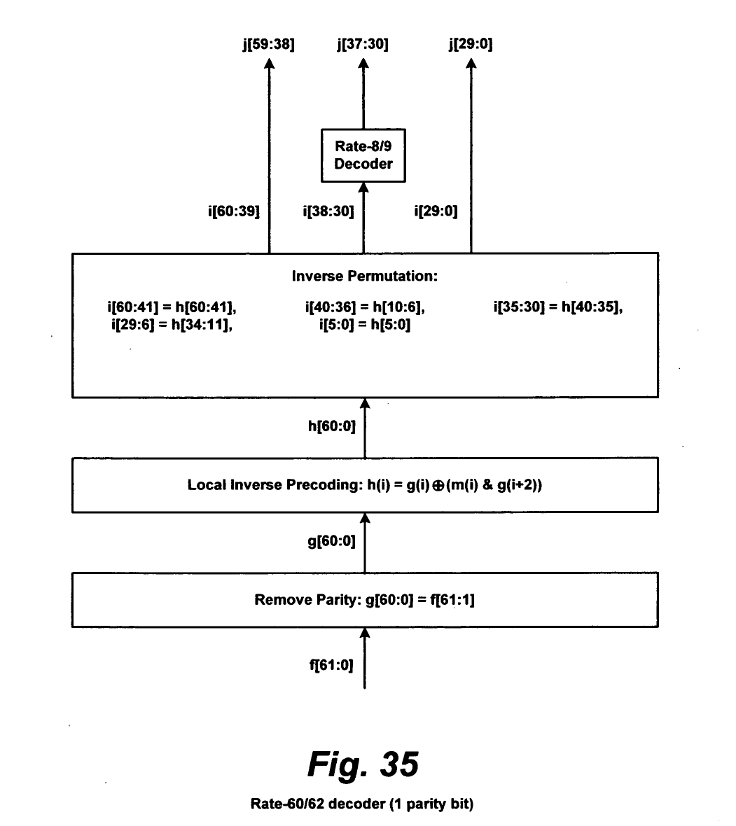

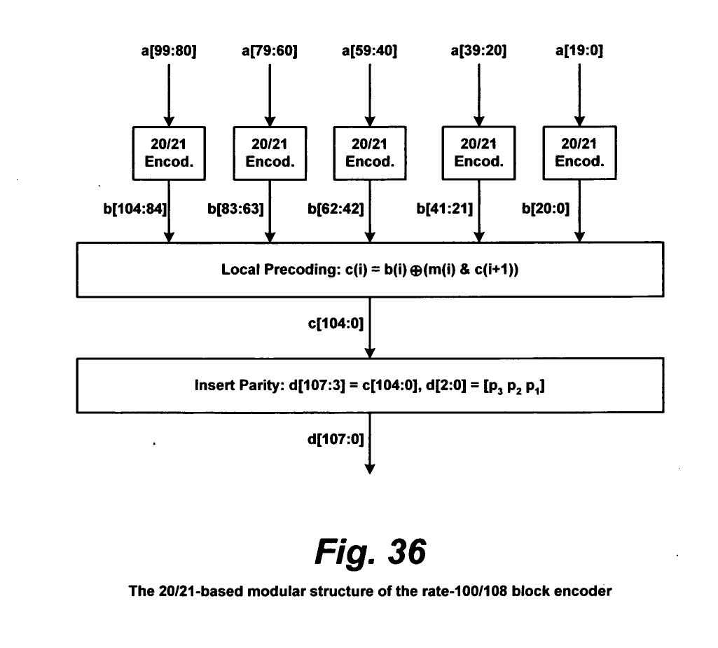

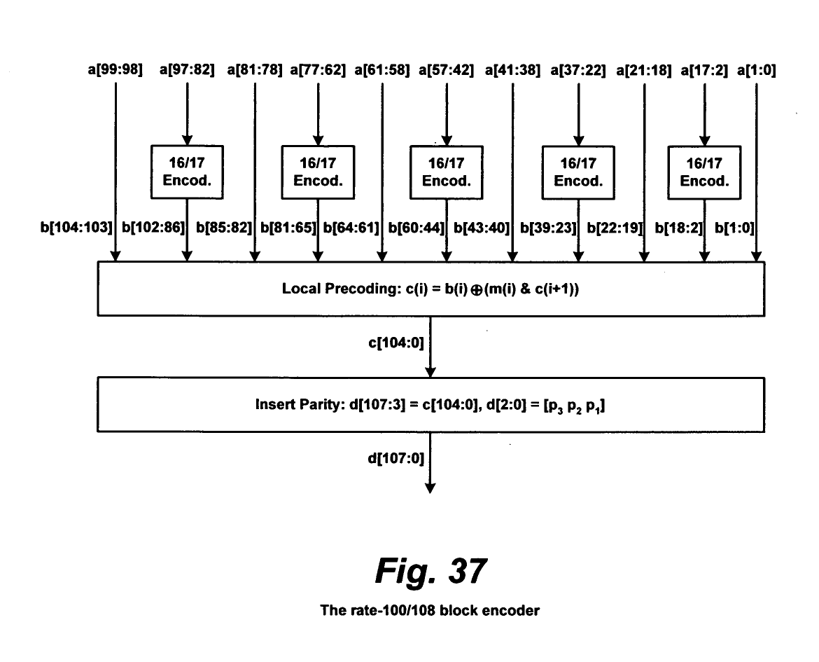

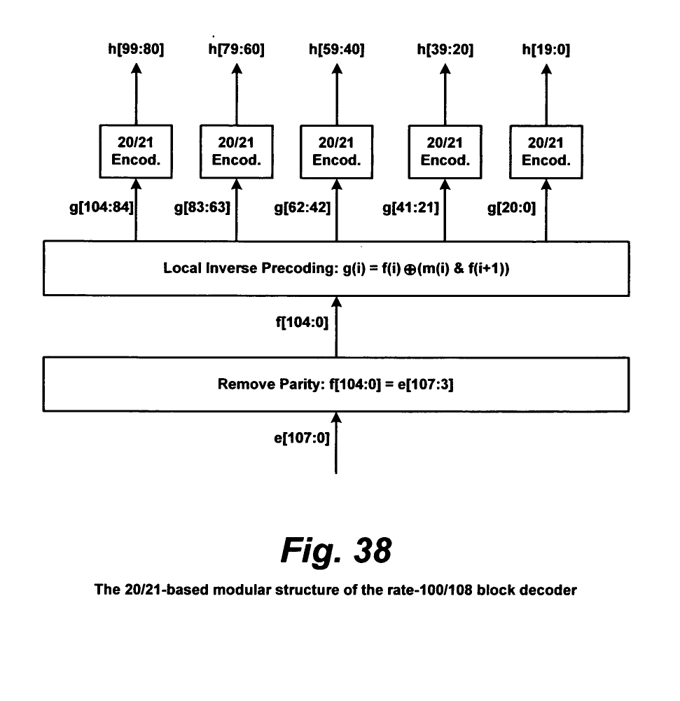

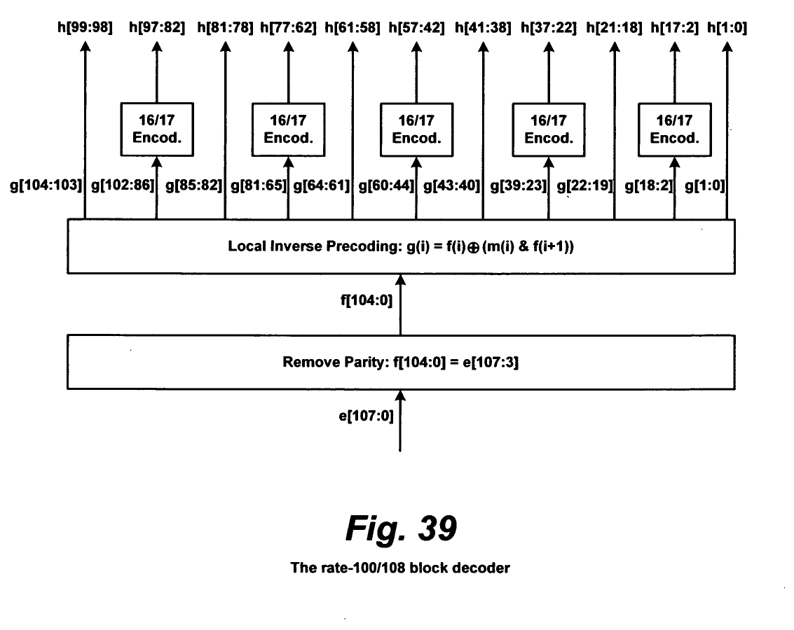

Now referring to FIG. 6a, there is provided a cervical plate 101 for providing dynamic stabilization of upper and lower cervical vertebrae. This plate is substantially similar to that shown in FIG. 1 above, except that the lateral axially-extending strut members 103 are bent inwards (instead of outwards, as in the device of FIG. 1). In this particular case, when the device of FIG. 6a is subject to an axial load, the strut members flex laterally inward (as shown in FIG. 6b). In preferred embodiments, the device of FIG. 6a is designed so that, when an extreme load is applied, the inward lateral movement of the inwardly flexing, axially extending strut members 103 cause these members to touch one another (as shown in FIG. 6b), thereby providing an effective stop against extreme movement.

Any conventional bone fastener may be used with the present invention, including threaded screws and anchors. In some embodiments (as in FIGS. 2-5b), a single screw is received by each of the single upper and lower transverse holes of the plate to provide the required fixation of the plate to the bone. In other embodiments (as in FIG. 1), there are a pair of transverse holes in each of the upper and lower portions of the plate, thereby requiring four screws for fixation.

Although each embodiment disclosed in the FIGS. is shown as a construct adapted for use with a single level discectomy or corpectomy procedure, the scope of the present invention also includes constructs adapted for use with multi-level discectomy or corpectomy procedures. In preferred embodiments thereof, the device is designed so that the device comprises a plurality of longitudinally elongated intermediate portions, each longitudinally elongated intermediate portion being adapted to provide independent motion at each level.

Also in accordance with the present invention, there is provided a novel method of implanting the device of the present invention. In this preferred method, the device is placed in an extended mode (e.g., loaded in axial tension) during insertion and fastening of the bone screws. Once the bone screws are securely fastened through the plate, the tension is released. Because the device is designed as so to avoid plastic deformation, the hole-to-hole spacing of the device returns to its unloaded value. This descrease in the hole-to-hole spacing also produces a desirable continuous compressive load on the graft site, thereby assisting in the fusion.

Therefore, in accordance with the present invention, there is provided a method of implanting an anterior cervical plate between adjacent vertebrae, comprising the steps of:
a) providing the plate of the present invention,
b) axially tensioning the device to produce an extended device length,
c) fastening the device to a pair of adjacent vertebrae, releasing the tension from the device.

Extension of the device can be accomplished in various conventional ways, provided it produces an adequate axial tension across the device. Such methods include using an instrument that squeezes the lateral aspects of the flexible zone together (i.e., lateral-to-medial force) or an instrument that pulls the hole spacing apart (i.e., axial tension force). In another embodiment, the device could be made of a shape memory metal having a relatively short length during the martensitic phase and a relatively longer length in the austenitic phase. The device of this embodiment would be implanted in its long length—martensitic phase. When the temperature of the device of this embodiment is raised to body temperature, the memory metal changes to its austenitic phase, thereby decreasing the length of the plate and applying a compressive load to the graft.

Therefore, there is provided a method of implanting an anterior cervical plate between adjacent vertebrae, comprising the steps of:
a) providing a plate of the present invention, the plate being made of a memory metal having a relatively long length during the martensitic phase and a relatively shorter length in the austenitic phase,
b) implanting the plate in its martensitic phase, raising the temperature of the plate to cause a shift to the austenitic phase, thereby decreasing the length of the plate and applying a compressive load to the graft.

In some embodiments, the device of the present invention is made of biocompatible metal such as a titanium alloy, cobalt-chormium alloy, or a stainless steel. However, in other embodiments, other non-metallic materials may be employed. In some embodiments, a plastic may be used as the material of construction. Plastics are generally less stiff than metals, and so are less prone to breakage. In some embodiments, a resorbable polymer may be used as the material of construction, thereby allowing the plate to be resorbed by the body after the fusion has taken place. In some embodiments, a composite material having a fiber phase may be used as the material of construction. The composite may provide anisotropic properties and produce a preferred orientation that could enhance the deflection characteristics of the device.

1. A cervical plate for providing dynamic stabilization of upper and lower cervical vertebrae, the plate having opposed inner and outer surfaces defining a transverse axis, and opposed upper and lower surfaces defining an elongated longitudinal axis, the plate comprising:
a) an upper portion having an upper transverse throughhole,
b) a lower portion having a lower transverse throughhole, and
c) a longitudinally elongated intermediate portion therebetween,
wherein the elongated portion is adapted to flex laterally under loading of the longitudinal axis.

2. The plate of claim 1 wherein the elongated intermediate portion has a curved portion.

3. The plate of claim 2 wherein the elongated intermediate portion has at least two curved portions.

4. The plate of claim 1 having a stiffness adapted to promote fusion of the upper and lower cervical vertebrae.

5. The plate of claim 2 wherein the curved portion is curved laterally.

6. The plate of claim 1 wherein the elongated intermediate portion comprises at least two lateral axially extending thin members, each lateral axially extending thin member extending from the upper portion to the lower portion and having a straight portion and a predetermined curve portion.

7. The plate of claim 1 wherein the elongated portion comprises:
i) a pair of lateral axially extending strut members, each member extending from the upper portion to the lower portion and having at least one axially extending closed slot therein, the slot defining a plurality of axially extending thin members within each axially extending strut member.

8. The plate of claim 7, wherein each member extending from the upper portion to the lower portion has a plurality of axially extending closed slots therein, the plurality of slots defining at least three axially extending thin members within each axially extending strut member.

9. The plate of claim 7 wherein the thin members comprise between about 10% and 40% of the width of the intermediate portion of the plate.

10. The plate of claim 7 wherein at least one thin member has a long edge and a short edge, wherein the long edge of the thin member extends in the anterior-posterior direction, and the short edge of the thin member extends in the medial-lateral direction.

11. The plate of claim 7 wherein at least one strut member comprises an upper portion having a first cross sectional portion, a lower portions having a second cross sectional portion, and a third cross-sectional portion therebetween, wherein the third cross-sectional portion has a reduced cross-section.

12. The plate of claim 1 wherein the elongated portion has a width and comprises:
i) a pair of lateral axially extending strut members, each member extending from the upper portion to the lower portion and defining a graft window therebetween, the graft window having a width comprising at least 30% of the width of the longitudinally elongated intermediate portion.

13. The plate of claim 1 wherein the elongated portion comprises a telescope unit.

14. The plate of claim 13 wherein the telescope unit comprises:
a) a rod extending from the upper portion and having a first end, and
b) a receiving tube extending into the lower portion and having a bore and a closed end surface,
wherein the rod is received in the bore.

15. The plate of claim 14 wherein the telescope unit is medially-located.

16. The plate of claim 1 further comprising:
c) a contour zone disposed between at least one of the upper and lower portions of the plate, and the longitudinally elongated intermediate portion.

17. The plate of claim 16 wherein the contour zone comprises a thinned section disposed between at least one of the upper or lower portions of the plate and the longitudinally elongated intermediate portion.

18. The plate of claim 1 wherein the elongated portion comprises a stop.

19. The plate of claim 1 wherein the elongated portion laterally flexes inward.

20. The plate of claim 1 wherein the elongated portion laterally flexes outward.

21. A method of implanting an anterior cervical plate between adjacent vertebrae, comprising the steps of:
a) providing the cervical plate,
b) axially tensioning the device to produce an extended device length,
c) fastening the device to a pair of adjacent vertebrae,
d) releasing the tension from the device.

22. The method of claim 21 wherein the axial tensioning step includes a step of laterally compressing the longitudinally elongated intermediate portion.

23. The method of claim 21 wherein the axial tensioning step includes a step of pulling the upper and lower portions of the plate in opposite directions.

24. A method of implanting an anterior cervical plate between adjacent vertebrae, comprising the steps of:
a) providing the cervical plate, the plate being made of a memory metal having a relatively long length during the martensitic phase and a relatively shorter length in the austenitic phase,
b) implanting the plate in its martensitic phase,
c) raising the temperature of the plate to cause a shift to the austenitic phase, thereby decreasing the length of the plate and applying a compressive load to the graft.
